(19) United States
(12) Patent Application Publication
(10) Pub. No.: US 2022/0354429 A1
(43) Pub. Date: Nov. 10, 2022

(54) MEDICAL DEVICE WITH SENSING CAPABILITIES

(71) Applicant: BOSTON SCIENTIFIC SCIMED, INC., Maple Grove, MN (US)

(72) Inventors: Kevin Windheuser, Hopkinton, MA (US); Geraldine Alice Toner, Raphoe (IE); Michael Rodgers, Mayo (IE); Carmel Ralph, Galway (IE); Dayna M. Pleau, South Attleboro, MA (US)

(73) Assignee: BOSTON SCIENTIFIC SCIMED, INC., Maple Grove, MN (US)

(21) Appl. No.: 17/736,654

(22) Filed: May 4, 2022

Related U.S. Application Data
(60) Provisional application No. 63/184,375, filed on May 5, 2021.

(51) Int. Cl. A61B 5/00; A61B 5/07

(52) U.S. Cl. CPC A61B 5/6862; A61B 5/076; A61B 2560/0219

(57) ABSTRACT

Example medical devices, including example stents and stent systems, are disclosed. An example stent includes an expandable tubular scaffold having a proximal end and a distal end, a first wire coupled to the tubular scaffold, wherein the first wire is shaped into a first coil. The example stent also includes a sensor electrically coupled to the first wire, wherein the sensor is inductively powered by a magnetic field passing through the first wire.

### CROSS REFERENCE TO RELATED APPLICATIONS

This application claims the benefit of and priority to U.S. Provisional Patent Application Ser. No. 63/184,375 filed on May 5, 2021, the disclosure of which is incorporated herein by reference.

### TECHNICAL FIELD

The present disclosure pertains to medical devices, and methods for manufacturing medical devices. More particularly, the present disclosure pertains to implantable medical devices and methods for manufacturing and using such devices.

### BACKGROUND

Implantable medical devices may incorporate a variety of features and/or components to wirelessly sense and/or transmit signals from remote locations within a patient. For example, certain stents may utilize a magnetic field source to inductively power a sensor coupled to the implanted stent. Other stents may utilize a dielectric polymer to power a sensor coupled to the implanted stent. Using a magnetic field to power various components of medical devices may be beneficial as magnetic fields may not be affected by other phenomena, such as mechanical contact, hydrodynamics, thermodynamics, etc. Additionally, a powered sensor may communicate wirelessly with a receiver (e.g., a receiver located within a handheld device) located at a remote location from the sensor. For example, a magnetically powered sensor may transmit one or more signals which may include information about physiological and/or anatomical characteristics of a patient (within which the stent is implanted), to a receiver located outside of the patient. Examples of powered medical devices and components thereof, such as powered stents and sensors, are disclosed herein.

### BRIEF SUMMARY

This disclosure provides design, material, manufacturing method, and use alternatives for medical devices. An example stent includes an expandable tubular scaffold having a proximal end and a distal end, a first wire coupled to the tubular scaffold, wherein the first wire is shaped into a first coil. The example stent also includes a sensor electrically coupled to the first wire, wherein the sensor is inductively powered by a magnetic field passing through the first wire.

Alternatively or additionally to any of the embodiments above, wherein the first wire is attached to an outer surface of the tubular scaffold.

Alternatively or additionally to any of the embodiments above, wherein the first wire is attached to an inner surface of the tubular scaffold.

Alternatively or additionally to any of the embodiments above, wherein the tubular scaffold further includes a plurality of braided filaments extending from the proximal end to the distal end, and wherein the first wire is included within the plurality of braided filaments.

Alternatively or additionally to any of the embodiments above, further comprising a second wire coupled to the tubular scaffold, wherein the second wire is shaped into a second coil, and wherein the first coil, the second coil or both the first coil and the second coil are attached to an outer surface of the tubular scaffold.

Alternatively or additionally to any of the embodiments above, wherein the sensor is configured to draw power from the first wire as the magnetic field passes through the first wire.

Alternatively or additionally to any of the embodiments above, wherein the sensor includes a battery configured to store the power being drawn from the first wire.

Alternatively or additionally to any of the embodiments above, wherein a signal transmitted by the sensor is configured to be received by a receiver located in a remote location from the sensor.

Alternatively or additionally to any of the embodiments above, wherein the sensor is selected from a group consisting of a temperature sensor, a pH sensor, a flow sensor, a pressure sensor, an oxygen sensor, and a heart rate sensor.

Alternatively or additionally to any of the embodiments above, wherein the sensor is attached to only the first wire.

Alternatively or additionally to any of the embodiments above, wherein the sensor is attached to a portion of the tubular scaffold, and wherein the tubular scaffold is configured to transfer power from the first wire to the sensor.

Alternatively or additionally to any of the embodiments above, wherein the first wire includes an insulated covering.

An example medical device system includes a magnetic field generator configured to generate a magnetic field and a stent. Further, the stent includes an expandable tubular scaffold having a proximal end, a distal end, and a lumen extending therethrough, a first wire coupled to the tubular scaffold, wherein the first wire is shaped into a coil, and a sensor electrically coupled to the first wire, wherein the sensor is inductively powered by the magnetic field passing through the first wire. The medical device further includes a receiver configured to receive signals transmitted by the sensor.

Alternatively or additionally to any of the embodiments above, wherein the first wire is attached to an outer surface of the tubular scaffold.

Alternatively or additionally to any of the embodiments above, wherein the first wire is coiled around the outer surface of the tubular scaffold along a majority of a length of the expandable tubular scaffold.

Alternatively or additionally to any of the embodiments above, wherein the tubular scaffold further includes a plurality of braided filaments extending from the proximal end to the distal end, and wherein the first wire is included within the plurality of braided filaments.

Alternatively or additionally to any of the embodiments above, wherein the magnetic field generator includes the receiver.

Alternatively or additionally to any of the embodiments above, wherein the magnetic field generator includes a handheld device

An example expandable medical device includes a tubular scaffold, the scaffold including an inner surface, an outer surface and a lumen extending therein. The medical device also includes a covering attached to the tubular scaffold, wherein the covering includes a dielectric elastomer. Further, the medical device includes a sensor electrically coupled to the dielectric elastomer. Additionally, the tubular scaffold is configured to deform from a first shape to a second shape, wherein deformation of the tubular scaffold from the first shape to the second shape deforms the dielectric elastomer, and wherein deformation of the dielectric elastomer provides power to the sensor.

Alternatively or additionally to any of the embodiments above, further comprising a battery configured to store electrical energy generated by deformation of the dielectric elastomer.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic diagram illustrating an example medical device inductively coupled to a magnetic field generated by a magnetic field generator;

FIG. 2 illustrates an example stent positioned within a magnetic field;

FIG. 3 illustrates an example stent including a coiled wire;

FIG. 4 is a cross-sectional view of a portion of the wire shown in FIG. 3;

FIG. 5 illustrates another example stent;

FIG. 6 illustrates a first step to couple a coiled wire to an implanted stent;

FIG. 7 illustrates a coiled wire coupled with the implanted stent shown in FIG. 6;

FIG. 8 illustrates another example stent in a first configuration;

FIG. 9 illustrates the example stent of FIG. 8 in a second configuration.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

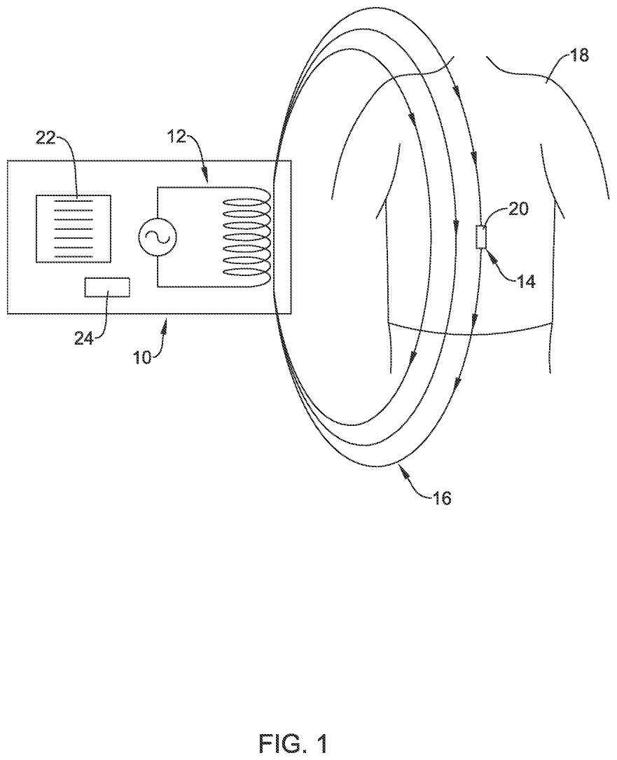

FIG. 1 illustrates an example magnetic field generator 10 positioned external of a patient 18. The magnetic field generator 10 may be coupled to a power source (e.g., an internal battery, an external battery or other external power source). Additionally, the magnetic field generator 10 may include one or more components configured to generate an electromagnetic field (represented by the closed magnetic field lines 16). For example, FIG. 1 illustrates that the magnetic field generator 10 may include a conductive wire 12 which is wound into a coil.

In some examples, the coiled wire 12 may be wound around a core. In some examples, the core around which the wire 12 is coiled may include a permanent magnet or a magnetic material. However, in other examples the core around which the wire 12 is coiled may not include a permanent magnet or magnetic material. The wire 12 may be tightly wound around the core, in which adjacent windings of the wire 12 are in contact with one another, or the wire 12 may be wound around the core such that adjacent windings of the wire 12 are spaced apart from one another.

FIG. 1 further illustrates that the coiled wire 12 may further be coupled to a voltage source. For example, magnetic field generator 10 may be configured to draw power from a power source (e.g., an internal battery or external power source) to generate an electrical current through the coiled wire 12. It can be appreciated that a coiled wire carrying an electric current will produce an electromagnetic field with closed field lines surrounding the wire (as illustrated in FIG. 1). For example, the coiled wire 12 carrying an electric current will produce a magnetic field with closed field lines 16 extending from the coiled wire 12.

It can be further appreciated that the electromagnetic field 16 generated by the magnetic field generator 10 may be increased (e.g., strengthened) by varying the configurations of the coiled wire 12 and/or the electrical current passing therethrough. For example, the electromagnetic field 16 may be increased by increasing the number of windings of the coiled wire 12. Additionally, the electromagnetic field 16 may be increased by increasing the thickness of the wire 12, while simultaneously increasing the electrical current passing therethrough. Additionally, another way the electromagnetic field 16 may be increased may be to lower the resistance in the wire 12 (by utilizing a more conductive wire, for example).

FIG. 1 further illustrates an implantable medical device 14 deployed within the patient 18. In some examples, the implantable medical device 14 may include an implantable stent 14. The implantable stent 14 may include a tubular scaffold having a first end, a second end, and a lumen extending from the first end to the second end. In some instances, the tubular scaffold may be formed of one or more, or a plurality of interwoven wires or filaments. For example, the tubular scaffold may be a knitted tubular structure formed of a single interwoven wire, or the tubular scaffold may be a braided tubular structure formed of a plurality of braided wires or filaments. In other instances, the tubular scaffold may be a monolithic structure having a plurality of struts defining interstices therebetween. However, it is contemplated that the medical device 14 may include other types of medical devices. For example, the medical device 14 may include a direct visualization device (e.g., a video capsule), endoscopy clips, tissue closure clips, etc. While FIG. 1 does not illustrate the stent 14 deployed in a particular body lumen and/or cavity, it is contemplated that the stent 14 may be utilized in a variety of body lumens and/or cavities. For example, it is contemplated that the stent 14 (and other example stents disclosed herein) may be utilized to treat the esophagus, large intestine, small intestine, peripheral arteries, coronary arteries, veins, neurovasculature or other similar body lumens and/or cavities.

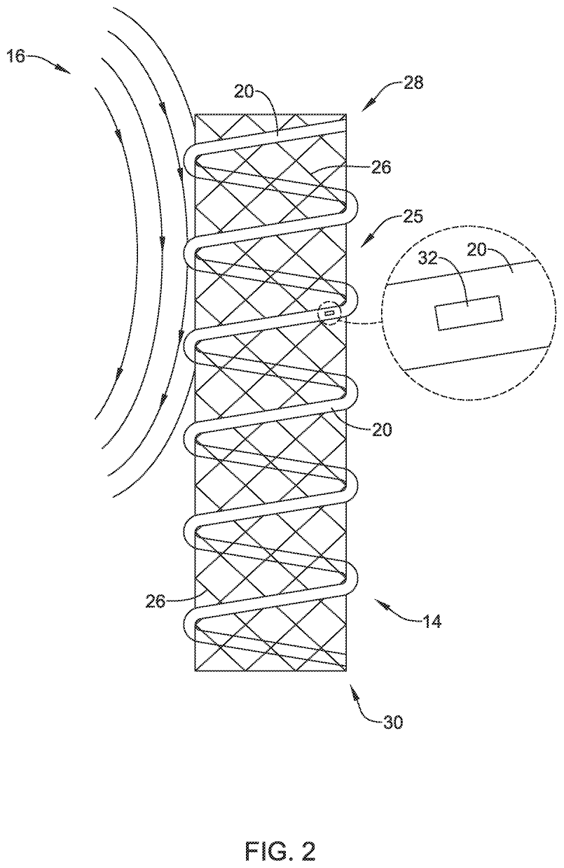

Additionally, FIG. 1 illustrates that the stent 14 may include a coiled wire 20. As will be described in greater detail below, when the wire 20 is positioned within range of the electromagnetic field 16 generated by the magnetic field generator 10 (e.g., the wire 20 is positioned such that the electromagnetic field lines 16 pass through the wire 20), an electric current may be generated through the wire 20. In this configuration, it can be appreciated that the coiled wire 12 and the coiled wire 20 are coupled via electromagnetic induction. For example, the wire 12 and the wire 20 may be inductively coupled (e.g., magnetically coupled) when they are arranged in space such that a change in current through the coiled wire 12 induces a voltage (e.g., creates a flow of electrons) through the coiled wire 20. As discussed herein, the coupling between the wire 12 and the wire 20 may be increased by winding each of them into coils and placing them close together on a common axis, such that the magnetic field of one of the coiled wire 12 or the coiled wire 20 passes through the other of the coiled wire 12 or the coiled wire 20, respectively. Further, as will be described in greater detail below, an electrical current generated within the wire 20 may be utilized to power a sensor 32 (shown in FIG. 2).

FIG. 1 further illustrates that the magnetic field generator 10 may include a receiver 24. The receiver 24 may be configured to receive signals sent by the sensor 32 (shown in FIG. 2). While FIG. 1 illustrates that the receiver 24 may be located within the magnetic field generator 10, it is also contemplated that the receiver 24 may be located in a separate device located outside of (e.g., spaced away from) the magnetic field generator 10. For example, in some instances the receiver 24 may be located in a handheld device (e.g., a mobile device, mobile tablet, mobile smart phone) or within a mobile workstation or computer.

Additionally, FIG. 1 illustrates that the magnetic field generator 10 may include a display 22. The display 22 may display signal information sent by the sensor 32 and received by the receiver 24. For example, the display 22 may provide a read out of physiological information of the patient 18 sensed by the sensor 32 and sent to the receiver 24. In some instances, the display 22 may be a touch screen display.

As described above, in some instances the receiver 24 may be located in a handheld device, a mobile workstation, a computer, etc. Therefore, it can be appreciated that any device which includes the receiver 24 may also include a display 22 which may display signal information sent by the sensor 32 and received by the receiver 24. For example, the display 22 located on a handheld device, a mobile workstation, a computer, etc. may provide a read out of physiological information of the patient 18 sensed by the sensor 32 and sent to the receiver 24. In some instances, the display 22 may be a touch screen display.

FIG. 2 illustrates the example stent 14 illustrated in FIG. 1. As described above, the example stent 14 may be positioned such that the electromagnetic field lines 16 generated by the coil 12 of the electromagnetic field generator 10 pass through the stent 14.

Further, FIG. 2 illustrates that the stent 14 may include a tubular scaffold 25. The tubular scaffold 25 may include a first end 28, a second end 30, and a lumen extending therethrough. Additionally, the tubular scaffold 25 may include a plurality of filaments and/or strut members 26 extending from the first end 28 to the second end 30. The filaments 26 may be arranged and/or engaged with one another in a variety of different arrangements and/or geometric patterns. In some examples, the filaments 26 may be laser cut from a unitary tubular member. In other examples, the filaments 26 may be one or more, or a plurality of wires braided, woven or constructed using a combination of these (or similar) manufacturing techniques. Therefore, numerous designs, patterns and/or configurations for the stent cell openings, strut thicknesses, strut designs, stent cell shapes are contemplated and may be utilized with embodiments disclosed herein.

The stent 14 may be delivered to a treatment area via a stent delivery system (not shown). For example, in some instances the stent 14 may be a balloon expandable stent. In some instances, balloon expandable stents may be manufactured from a single, cylindrical tubular member (e.g., a cylindrical tubular member may be laser cut to form a balloon expandable stent).

In other examples, the stent 14 may be a self-expanding stent. A self-expanding stent may be delivered to a treatment area in a radially constrained configuration via a self-expanding stent delivery system, and then released from the stent delivery system to radially expand automatically to a deployed configuration when unconstrained by the stent delivery system. It is contemplated that the examples disclosed herein may be utilized with any one of various stent configurations, including, balloon expandable stents, such as a laser cut stent and/or a braided stent, a self-expanding stent, non-expandable stents, or other stents.

Additionally, the stent filaments 26 disclosed herein may be constructed from a variety of materials. For example, the filaments 26 may be constructed from a metal (e.g., Nitinol). In other instances, the filaments 26 may be constructed from a polymeric material (e.g., PET). In yet other instances, the filaments 26 may be constructed from a combination of metallic and polymeric materials. Further, the filaments 26 may include a bioabsorbable and/or biodegradable material.

As described above, FIG. 2 further illustrates that the stent 14 may include a coiled wire 20 which may extend from the first end 28 to the second end 30 of the stent 14. While FIG. 2 illustrates the wire 20 extending from the first end 28 to the second end 30 of the stent 14, it is contemplated that the wire 20 may extend only along a portion of the length of the stent 14.

Further, in some examples (such as that shown in FIG. 2), the coiled wire 20 may be helically wrapped around an outer surface of the tubular scaffold 25 to form a coiled configuration. However, in other examples, the coiled wire 20 may be positioned within the lumen of the tubular scaffold 25, whereby it may be helically wrapped around an inner surface of the tubular scaffold 25 to form a coiled configuration. In some instances, the adjacent windings of the coiled wire 20 may be spaced apart from one another, forming an open wound coil. In other instances, the adjacent windings of the coiled wire 20 may be in contact with one another, forming a closed wound coil. Further, in some examples, the first end of the wire 20 may be attached to a second end of the wire 20, forming a continuous, uninterrupted coil attached to the outer surface of the tubular scaffold 25.

Additionally, the detailed view of FIG. 2 illustrates that the stent 14 may include a sensor 32 attached to the wire 20. In some examples, the sensor 32 may be attached to an outer surface of the wire 20. Further, it can be appreciated that the sensor 32 may be electrically attached to the wire 20 such that a current passing through the wire 20 may power the sensor 32.

For example, as described above, the wire 20 may be inductively coupled to the wire 12, such that the electromagnetic field 16 generated by an electric current passing through the coil 12 may create an electrical current within the wire 20, which, in turn, may power the sensor 32. Once the sensor 32 is powered, it may sense one or more physiological parameters of the patient, performance characteristics of the stent, movement of the stent, etc. and transmit a signal representing the one or more parameters and/or stent characteristics to the receiver 24 located in the electromagnetic generator 10. It can be appreciated that powering the sensor 32 via inductive coupling may eliminate the need for the sensor 32 to have a battery, thereby permitting the sensor 32 to be designed with a smaller footprint compared to a sensor which requires a battery to operate. Therefore, in some instances, the implanted stent 14 and associated sensor 32 may be devoid of a battery or other power storage component. However, in other instances, the wire 20 may be electrically coupled to a battery provided with the sensor 32 and/or stent 14 to store electrical energy to power the sensor 32, if desired.

It can be appreciated that the sensor 32 may include a variety of types of sensors designed to sense a variety of physiological parameters. For example, the sensor 32 may include a temperature sensor, pH sensor, a flow sensor, a pressure sensor, an oxygen sensor, a heart rate sensor, proximity sensor, an accelerometer, etc. Further, the sensor 32 may be configured to sense physiological parameters such as body temperature, pH levels, blood pressure, blood flow rate, oxygen saturation levels, heart rate, etc.

It can be appreciated that, in some instances, the sensor 32 and/or the stent 14 may include a battery which may be used to store the energy generated in the wire 20. For example, the sensor 32 and/or the stent 14 may include a battery which is coupled to the wire 20 (e.g., electrically coupled to the wire 20), whereby by the battery may draw and store power from the electrical current passing through the wire 20. Additionally, it can be appreciated that power saved in the battery may be utilized to power the sensor 32 when the wire 20 is not inductively coupled to the wire 12.

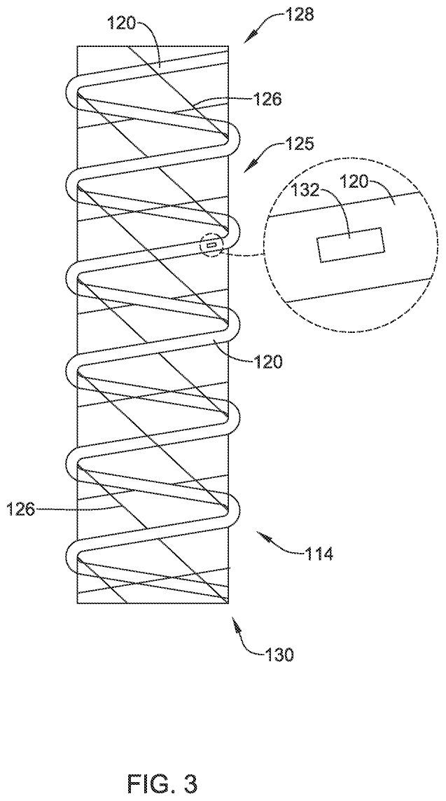

FIG. 3 illustrates another example stent 114. The example stent 114 may be similar in form and function as the stent 14 described above. For example, the stent 114 may include a tubular scaffold 125. The tubular scaffold 125 may include a first end 128, a second end 130, and a lumen extending therethrough. Additionally, the tubular scaffold 125 may include a plurality of filaments and/or strut members 126 extending from the first end 128 to the second end 130. The filaments 126 may be arranged and/or engaged with one another in a variety of different arrangements and/or geometric patterns. In some examples, the filaments 126 may be laser cut from a unitary tubular member. In other examples, the filaments 126 may be one or more, or a plurality of wires braided, woven or constructed using a combination of these (or similar) manufacturing techniques. Therefore, numerous designs, patterns and/or configurations for the stent cell openings, strut thicknesses, strut designs, stent cell shapes are contemplated and may be utilized with embodiments disclosed herein.

The stent 114 may be delivered to a treatment area via a stent delivery system (not shown). For example, in some instances the stent 114 may be a balloon expandable stent. In some instances, balloon expandable stents may be manufactured from a single, cylindrical tubular member (e.g., a cylindrical tubular member may be laser cut to form a balloon expandable stent).

In other examples, the stent 114 may be a self-expanding stent. A self-expanding stent may be delivered to a treatment area in a radially constrained configuration via a self-expanding stent delivery system, and then released from the stent delivery system to radially expand automatically to a deployed configuration when unconstrained by the stent delivery system. It is contemplated that the examples disclosed herein may be utilized with any one of various stent configurations, including, balloon expandable stents, such as a laser cut stent and/or a braided stent, a self-expanding stent, non-expandable stents, or other stents.

Additionally, the stent filaments 126 disclosed herein may be constructed from a variety of materials. For example, the filaments 126 may be constructed from a metal (e.g., Nitinol). In other instances, the filaments 26 may be constructed from a polymeric material (e.g., PET). In yet other instances, the filaments 126 may be constructed from a combination of metallic and polymeric materials. Further, the filaments 126 may include a bioabsorbable and/or biodegradable material.

Additionally, FIG. 3 further illustrates that the stent 114 may include a coiled wire 120 which may extend from the first end 128 to the second end 130 of the stent 114. While FIG. 3 illustrates the wire 120 extending from the first end 128 to the second end 130 of the stent 114, it is contemplated that the wire 120 may extend only along a portion of the length of the stent 114.

Further, in some examples (such as that shown in FIG. 3), the wire 120 may be one of the plurality of filaments or struts 126 which are utilized to construct the tubular scaffold 125. For example, the wire 120 may be braided and/or interwoven with one or more filaments 126 to construct the braided tubular scaffold 125. FIG. 3 illustrates that the wire 120 passing over/under one or more filaments 126 to construct the braided tubular scaffold 125. In other examples, the wire 120 may be passed through interstices which are formed between individual struts 126 defining the tubular scaffold 125. It can be further appreciated that the stent 114 may be designed to maintain the wire 120 in a coiled shaped despite being braided, interwoven or otherwise integrated with the stent filaments and/or struts 126. For example, the wire 120 may be braided and/or interwoven along a coiled path through a plurality of braided and/or interwoven filaments 126. Similarly, the wire 120 may be wound through interstitial spaces along a coiled path between stent filaments 126.

Additionally, the detailed view of FIG. 3 illustrates that the stent 114 may include a sensor 132 attached to the wire 120. The sensor 132 may be similar in form and function to the sensor 32 described above. For example, in some examples, the sensor 132 may be attached to an outer surface of the wire 120. Further, it can be appreciated that the sensor 132 may be electrically attached to the wire 120 such that a current passing through the wire 120 may power the sensor 132. For example, as described above, the wire 120 may be inductively coupled to the wire 12, such that an electromagnetic field generated by an electric current passing through the coil 12 may create an electrical current within the wire 120, which, in turn, may power the sensor 132. Once the sensor 132 is powered, it may sense one or more physiological parameters (as described above) and transmit a signal representing the one or more parameters to the receiver 24 located in the electromagnetic generator 10.

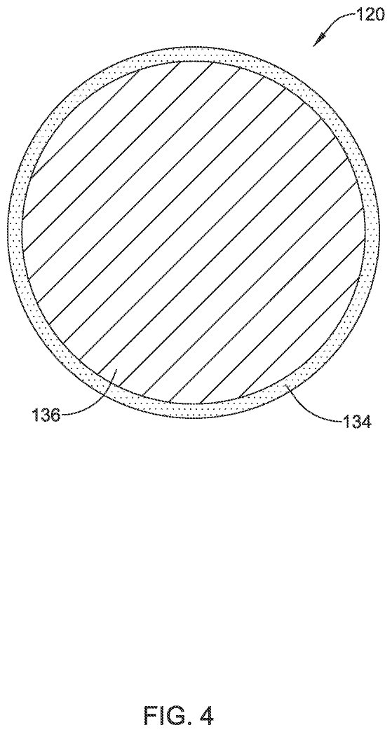

FIG. 4 illustrates a cross-section of the wire 120. It is noted that the cross-section illustrated in FIG. 4 may also represent a cross-section of the wire 20 described above. FIG. 4 illustrates that the cross-section of the wire 120 may include an inner core 136. The inner core 136 may be formed from a conductive metal (e.g., copper, silver, gold, etc.). FIG. 4 further illustrates that the wire 120 may include a non-conductive, insulated cover 134 surrounding the inner core 136. In some instance, the insulated cover 134 may be formed of a polymeric material, while the conductive core 136 may be formed of a metallic material.

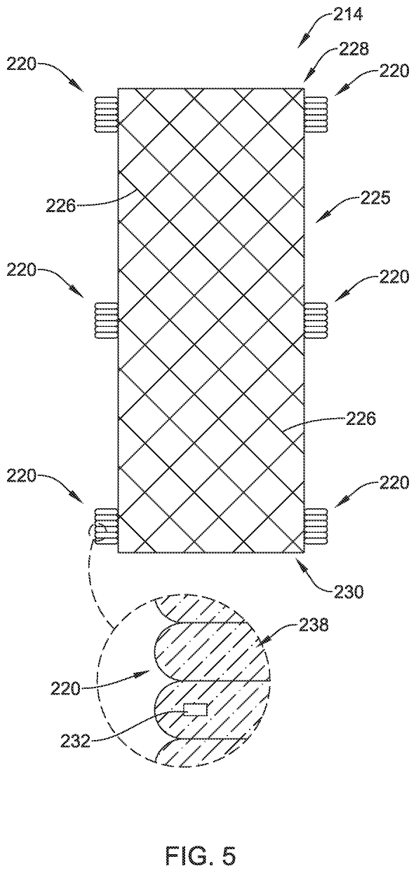

FIG. 5 illustrates another example stent 214. The example stent 214 may be similar in form and function as other stents described herein. For example, the stent 214 may include a tubular scaffold 225. The tubular scaffold 225 may include a first end 228, a second end 230, and a lumen extending therethrough. Additionally, the tubular scaffold 225 may include a plurality of filaments and/or strut members 226 extending from the first end 228 to the second end 230. The filaments 226 may be arranged and/or engaged with one another in a variety of different arrangements and/or geometric patterns. In some examples, the filaments 226 may be laser cut from a unitary tubular member. In other examples, the filaments 226 may be one or more, or a plurality of wires braided, woven or constructed using a combination of these (or similar) manufacturing techniques. Therefore, numerous designs, patterns and/or configurations for the stent cell openings, strut thicknesses, strut designs, stent cell shapes are contemplated and may be utilized with embodiments disclosed herein.

Additionally, FIG. 5 illustrates that the stent 214 may include one or more coiled wires 220 attached to the outer surface of the tubular scaffold 225. It can be appreciated from FIG. 5 that each of the plurality of coiled wires 220 may be relatively small compared to the tubular scaffold 225. Additionally, it can be further appreciated that each of the coiled wires 220 may be electrically coupled to one another, whereby the combination of smaller coiled wires 220 may include performance characteristics which are similar to the larger wires 20/120 described herein. For example, the combined windings of the combined smaller coiled wires 220 may be similar in form and function to the single coiled wires 20/120 described herein.

Further, the each of the smaller coiled wires 220 may be longitudinally aligned with one another from the first end 228 to the second end 230 of the tubular scaffold 225. However, it is contemplated that, in other examples, the plurality of coiled wires 220 may not be longitudinally aligned along the outer surface of the tubular scaffold 225. Rather, it is contemplated that the plurality of coiled wires 220 may be spaced along the outer surface of the tubular scaffold 225 in any type of arrangement. For example, the plurality of coils may be spaced irregularly along the outer surface of the tubular scaffold 225. In some instances, the plurality of coils may be arranged at a plurality of circumferential locations around the circumference of the tubular scaffold 225. For example, the plurality of coiled wires 220 may be uniformly spaced, or non-uniformly spaced around the circumference, if desired. In some instances, each of the plurality of coiled wires 220 may extend along a majority of the length of the tubular scaffold 225 at circumferentially spaced apart locations. It can be appreciated that when the stent 214 is deployed in a body lumen, the placement of the plurality of coiled wires 220 on the outside of the tubular scaffold 225 may contact the body lumen, thereby improving the stent's ability to maintain its position in the lumen (e.g., reduce the likelihood the stent will migrate).

Further, the detailed view of FIG. 5 illustrates that the stent 214 may include a sensor 232 attached to one of the coiled wires 220. The sensor 232 may be similar in form and function to other sensors described herein. For example, the sensor 232 may be attached to an outer surface of one of the coiled wires 220. Further, it can be appreciated that the sensor 232 may be electrically attached to one of the coiled wires 220 such that a current passing through the wires 220 may power the sensor 232. For example, the wires 220 may be inductively coupled to the wire 12 (shown in FIG. 1), such that an electromagnetic field generated by an electric current passing through the coil 12 may create an electrical current within the wires 220, which, in turn, may power the sensor 232. Once the sensor 232 is powered, it may sense one or more physiological parameters and transmit a signal representing the one or more parameters to the receiver 24 located in the electromagnetic generator 10.

Additionally, the detailed view of FIG. 5 further illustrates that, in some examples, one or more of the coiled wires 220 may include an insulated covering 238 (e.g., a non-conductive insulated covering like the covering 134 described herein). However, the detailed view of FIG. 5 further illustrates that the outer surface of the insulated covering 238 may further include a patterned and/or roughened surface texture (depicted by the dashed lines in the detailed view of FIG. 5). It can be appreciated that when the stent 214 is deployed in a body lumen, the roughened surface texture of the insulated covering 238 may increase friction between the coiled wires 220 and the body lumen, thereby improving the stent's ability to maintain its position in the lumen (e.g., reduce the likelihood the stent will migrate).

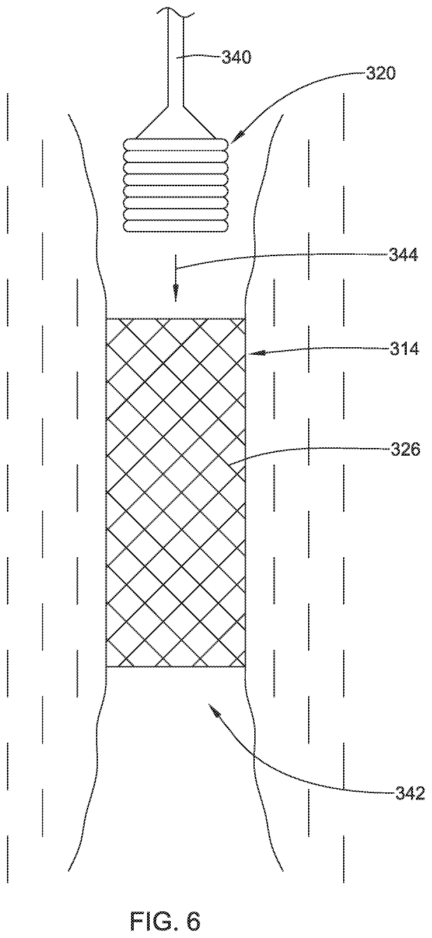

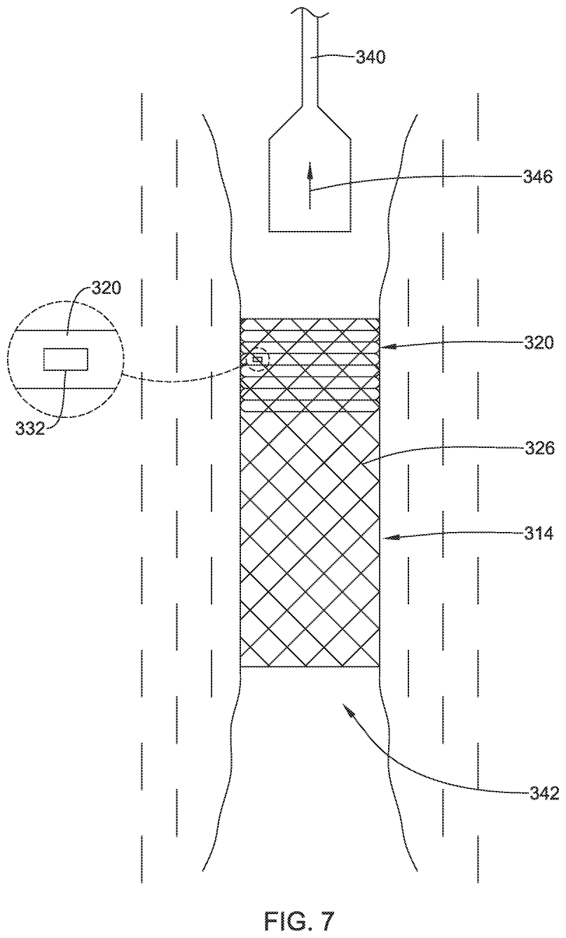

FIGS. 6-7 illustrates that, in some examples, an inductive coil may be delivered and coupled to a previously deployed stent. For example, FIG. 6 illustrates an example stent 314 deployed in a body lumen 342. It can be appreciated that upon its initial deployment into the body lumen 342, the stent 314 may not include a coiled wire or a sensor coupled thereto, and therefore, may not sense nor communicate any sensed physiological parameters, stent characteristics, etc. to a remote receiver. Therefore, in some instances, it may be beneficial to add a coiled wire including a sensor to the stent 314 at a later point in time, such as subsequent to implanting the stent 314 during a medical procedure or during a subsequent medical procedure after a previous medical procedure in which the stent was implanted.

Accordingly, FIG. 6 illustrates a coiled wire 320 releasably attached to an outer surface of a coiled wire deployment device 340. FIG. 6 further illustrates the coiled wire deployment device 340 being advanced toward the stent 314 (advancement of the deployment device 340 is depicted by the arrow 344). It can be appreciated that the coiled wire 320 may be attached to the deployment device 340 by one or more features which may release the coiled wire 320 after the coiled wire 320 is positioned within the inner lumen of the stent 314. The coiled wire 320 may be held and delivered by the deployment device 340 in a radially constrained, smaller diameter configuration, and then released within the lumen of the stent 314 to radially expand to a larger diameter configuration. For example, the coiled wire 320 may be a singular, separate coil configured to radially expand upon being released from the coiled wire deployment device 340, whereby the radial expansion of the coiled wire 320 provides a radial force sufficient to couple the coiled wire 320 to the inner surface of the stent 314.

While the coiled wire deployment device 340 described above includes the coiled wire 320 positioned along an outer surface of the deployment device 340 in a radially constrained configuration, other configurations are contemplated. For example, in some configurations, the deployment device 340 may include a retractable sheath which may house the coiled wire 320 in a radially constrained configuration as the deployment device is advanced to the stent 314. It can be further appreciated that after the sheath is positioned within the lumen of the stent 314, the sheath may be retracted, thereby permitting the coiled wire 320 to radially expand and couple to the inner surface of the stent 314.

FIG. 7 illustrates the example stent 320 deployed within the inner lumen of the stent 314. Further, FIG. 7 illustrates the coiled wire deployment device 340 being retracted away from the stent 314 after having released the coiled wire 314 (retraction of the coiled wire deployment device is depicted by the arrow 346 in FIG. 7).

It can further be appreciated that the coiled wire 320 illustrated in FIG. 7 may include a sensor 332 attached to the coiled wire 320. The sensor 332 may be similar in form and function to other sensors described herein. For example, the sensor 332 may be attached to an outer surface of the coiled wire 320. Further, it can be appreciated that the sensor 332 may be electrically attached to the coiled wire 320 such that a current passing through the wire 320 may power the sensor 332. For example, the wire 320 may be inductively coupled to the wire 12 (shown in FIG. 1), such that an electromagnetic field generated by an electric current passing through the coil 12 may create an electrical current within the wire 320, which, in turn, may power the sensor 332. Once the sensor 332 is powered, it may sense one or more physiological parameters and transmit a signal representing the one or more parameters to the receiver 24 located in the electromagnetic generator 10.

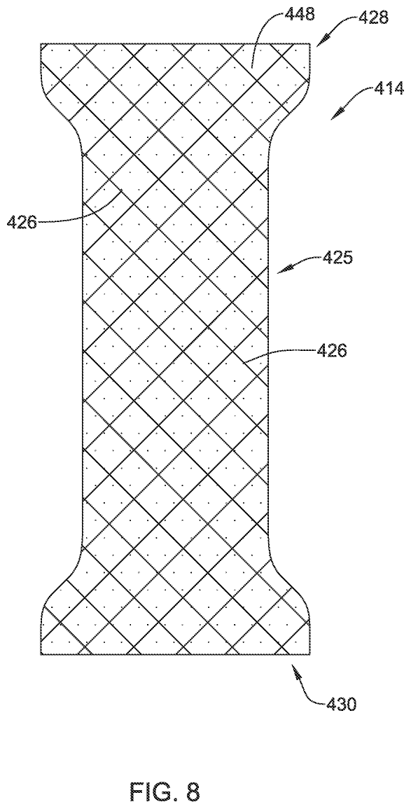

FIG. 8 illustrates another example stent 414. The example stent 414 may be similar in form and function as other stents described herein. For example, the stent 414 may include a tubular scaffold 425. The tubular scaffold 425 may include a first end 428, a second end 430, and a lumen extending therethrough. Additionally, the tubular scaffold 425 may include a plurality of filaments and/or strut members 426 extending from the first end 428 to the second end 430. The filaments 426 may be arranged and/or engaged with one another in a variety of different arrangements and/or geometric patterns. In some examples, the filaments 426 may be laser cut from a unitary tubular member. In other examples, the filaments 426 may be one or more, or a plurality of wires braided, woven or constructed using a combination of these (or similar) manufacturing techniques. Therefore, numerous designs, patterns and/or configurations for the stent cell openings, strut thicknesses, strut designs, stent cell shapes are contemplated and may be utilized with embodiments disclosed herein.

Additionally, FIG. 8 illustrates that the stent 414 may include a polymeric covering 448 (e.g., a polymeric layer or coating) extending from the first end 428 to the second end 430 of the tubular scaffold 425. While FIG. 8 illustrates the covering 448 extending from the first end 428 to the second end 430 of the tubular scaffold 425, it is contemplated that, in other examples, the covering 448 may extend along only a portion the length of the tubular scaffold 425. Additionally, it can be appreciated that the polymeric covering 448 may be positioned along the outer surface of the tubular scaffold 425, the inner surface of the tubular scaffold 425 or along both the outer surface of the tubular scaffold 425 and the inner surface of the tubular scaffold 425.

It can be appreciated that, in some examples, the polymeric covering 448 may include a dielectric polymer. It can be appreciated that a deformation incited upon a dielectric polymer may result in a flow of electrons through the dielectric polymer and any wire, electrodes, etc. coupled to the dielectric polymer. It can be further appreciated that the power generated by the dielectric polymer may be used to power a sensor positioned on the stent 414. For example, a sensor, such as a pressure sensor, temperature sensor, flow sensor, pH level sensor, oxygen sensor, etc. may be attached to the stent and electrically connected to the dielectric polymer such that electrical current generated by the cyclic deformation of the dielectric polymer flows to the sensor to power the sensor. Additionally, the power generated by the dielectric polymer may be stored in a battery and utilized as a reserve power source for a sensor positioned on the stent 414. The battery or other power storage element may be electrically connected to the dielectric polymer such that electrical current generated by the cyclic deformation of the dielectric polymer flows to the battery to be stored for later use in powering a sensor attached to the stent 414.

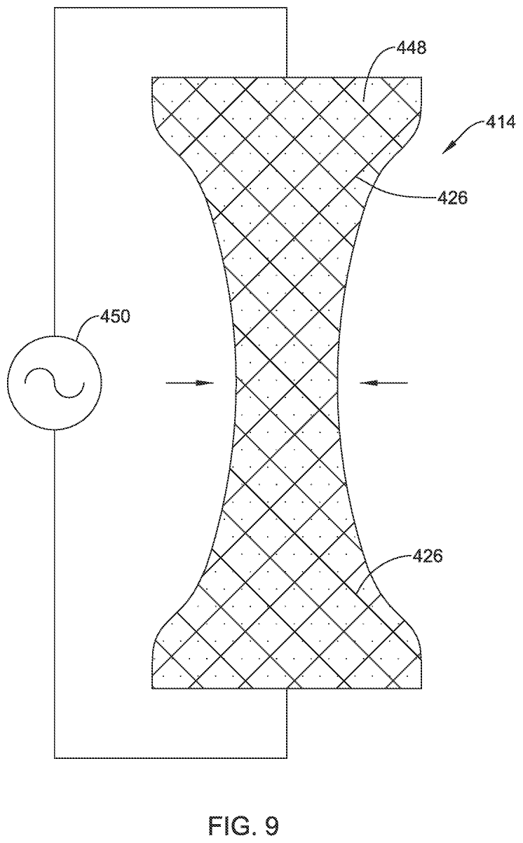

As discussed above, a voltage may be generated by the dielectric polymer upon deformation of the dielectric polymer. Accordingly, FIG. 9 illustrates the stent 414 being deformed into a second position relative to its position illustrated in FIG. 8. Further, FIG. 9 illustrates a voltage 450 being generated by the deformation of the polymeric coating 448 which includes a dielectric polymer.

An instance in which the stent 414 may undergo a deformation (such as a cyclic or repeated deformation similar to that illustrated in FIGS. 8-9) may occur when the stent 414 is positioned in a body lumen which exposes the stent 414 to peristaltic contractions. Peristaltic contractions are involuntary wave-like contractions which serve to propogate digested material (e.g., food) through the gastrointestinal tract. Accordingly, it can be appreciated that placing the stent 414 in a body lumen which undergoes peristaltic contractions may result in repeated or cyclic deformation of the stent 414. As discussed above, the repeated or cyclic deformation of the dielectric polymer in the polymeric coating 448 of the stent 414 may generate an electric current which may power a sensor and/or be stored in a battery or similar structure attached to the stent 414.

It can be further appreciated that the dielectric polymer in the polymeric coating 448 may be thin (e.g., include a substantially low-profile) and flexible to permit the stent 414 to move freely. Additionally, the dielectric polymer may include a variety of sizes, depending on its desired application and power output required. Further, in some examples, the dielectric polymer may cover all (or a substantial portion of) the stent 414 to limit tissue ingrowth. This may permit the stent to be removable from the body. However, in other examples, the dielectric polymer may cover only selected portions of the stent 414, which may permit tissue ingrowth to occur.

The materials that can be used for the various components of the medical device 10 and the various other medical devices disclosed herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 and the various other medical devices disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 and the various other medical devices disclosed herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 and the various other medical devices disclosed herein to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent, comprising:
an expandable tubular scaffold having a proximal end and a distal end;
a first wire coupled to the tubular scaffold, wherein the first wire is shaped into a first coil; and
a sensor electrically coupled to the first wire;
wherein the sensor is inductively powered by a magnetic field passing through the first wire.

2. The stent of claim 1, wherein the first wire is attached to an outer surface of the tubular scaffold.

3. The stent of claim 1, wherein the first wire is attached to an inner surface of the tubular scaffold.

4. The stent of claim 1, wherein the tubular scaffold further includes a plurality of braided filaments extending from the proximal end to the distal end, and wherein the first wire is included within the plurality of braided filaments.

5. The stent of claim 1, further comprising a second wire coupled to the tubular scaffold, wherein the second wire is shaped into a second coil, and wherein the first coil, the second coil or both the first coil and the second coil are attached to an outer surface of the tubular scaffold.

6. The stent of claim 1, wherein the sensor is configured to draw power from the first wire as the magnetic field passes through the first wire.

7. The stent of claim 6, wherein the sensor includes a battery configured to store the power being drawn from the first wire.

8. The stent of claim 1, wherein a signal transmitted by the sensor is configured to be received by a receiver located in a remote location from the sensor.

9. The stent of claim 1, wherein the sensor is selected from a group consisting of a temperature sensor, a pH sensor, a flow sensor, a pressure sensor, an oxygen sensor, and a heart rate sensor.

10. The stent of claim 1, wherein the sensor is attached to only the first wire.

11. The stent of claim 1, wherein the sensor is attached to a portion of the tubular scaffold, and wherein the tubular scaffold is configured to transfer power from the first wire to the sensor.

12. The stent of claim 1, wherein the first wire includes an insulated covering.

13. A medical device system, comprising:
a magnetic field generator configured to generate a magnetic field;
a stent, comprising:
an expandable tubular scaffold having a proximal end, a distal end, and a lumen extending therethrough;
a first wire coupled to the tubular scaffold, wherein the first wire is shaped into a coil; and
a sensor electrically coupled to the first wire, wherein the sensor is inductively powered by the magnetic field passing through the first wire; and
a receiver configured to receive signals transmitted by the sensor.

14. The stent of claim 13, wherein the first wire is attached to an outer surface of the tubular scaffold.

15. The medical device system of claim 14, wherein the first wire is coiled around the outer surface of the tubular scaffold along a majority of a length of the expandable tubular scaffold.

16. The stent of claim 13, wherein the tubular scaffold further includes a plurality of braided filaments extending from the proximal end to the distal end, and wherein the first wire is included within the plurality of braided filaments.

17. The medical device system of claim 13, wherein the magnetic field generator includes the receiver.

18. The medical device system of claim 17, wherein the magnetic field generator includes a handheld device.

19. An expandable medical device, comprising:
a tubular scaffold, the scaffold including an inner surface, an outer surface and a lumen extending therein; and
a covering attached to the tubular scaffold, wherein the covering includes a dielectric elastomer;
a sensor electrically coupled to the dielectric elastomer;
wherein the tubular scaffold is configured to deform from a first shape to a second shape;
wherein deformation of the tubular scaffold from the first shape to the second shape deforms the dielectric elastomer; and
wherein deformation of the dielectric elastomer provides power to the sensor.

20. The medical device of claim 19, further comprising a battery configured to store electrical energy generated by deformation of the dielectric elastomer.
